(19) United States
(12) Patent Application Publication
(10) Pub. No.: US 2015/0018691 A1
(43) Pub. Date: Jan. 15, 2015

(54) DEVICE FOR IMAGING, RECORDING AND SAVING THERMOGRAPHIC IMAGE, A SYSTEM OF THREE LIQUID CRYSTAL MATRICES USED BY THIS DEVICE AND ITS APPLICATION FOR THE DETECTION OF THERMAL ANOMALIES, AND A METHOD OF DIAGNOSING THESE ANOMALIES

(71) Applicant: BRASTER SA, Ozarów Mazowiecki (PL)

(72) Inventors: Jacek Bernard Stepien, Warszawa (PL); Henryk Jaremek, Warszawa (PL); Grzegorz Franciszek Pielak, Serock (PL)

(73) Assignee: BRASTER SA, Ozarow Mazowiecki (PL)

(21) Appl. No.: 14/376,788

(22) PCT Filed: Feb. 6, 2013
(86) PCT No.: PCT/IB2013/050990

§ 371 Date: Aug. 5, 2014

(30) Foreign Application Priority Data
Feb. 6, 2012 (PL) PL398030

(51) Int. Cl. A61B 5/00; A61B 5/01

(52) U.S. Cl. CPC A61B 5/0091; A61B 5/015
USPC 600/474

(57) ABSTRACT

The invention relates to a device for imaging, recording and saving a thermographic image of a breast, characterized in that it comprises a video recorder and an infrared mapping liquid crystal matrix working in the temperature range of approximately 1° in a range of 31.8° C. to 34.8″ C., wherein the recorder preferably includes a housing, a light source, a camera with an optoelectronic transducer, an analog-digital converter, a power source and a storage medium. The present invention also refers to a method of diagnosing of the breast pathology using that device, a system of the three infrared mapping liquid crystal matrices and its application for the detection of thermal anomalies on the surface of the breast.

The present invention refers to a device for imaging, recording and saving thermographic image of breast surface in women and a method of diagnosing of thermal anomalies. The present invention refers also to the system of three liquid crystal matrices used by this device and its application for the detection of these thermal anomalies.

The present invention is used for the detection of pathophysiological processes taking place in the breast in women during the periods except the lactation, which have specific and unique thermodynamic characteristics associated with adequate expression of heat. Degenerative processes taking place in the breast, such as fibro-cystic degeneration (degeneratio fibrocistica) due to regional restrictions of the blood supply are characterised by reduced temperature in relation to the surrounding tissue, which is associated with the hypothermic expression on the surface of the examined breast. On the other hand, according to the experimental studies [Zhao at al., (Qi Zhao, Jiaming Zhang, Ru Wang, Wei Cong, Use of a Malignant Tumour Thermocouple for detection, IEEE Eng. In Medicine and Biology Mag., January/February 2008)] the proliferative (neoplastic) processes are associated with the neoangiogenesis giving intraorganic hyperthermia focus, which may be monitored and recorded on the surface of the examined breast, as the areas of increased temperature relative to the surrounding tissue. The use of two thermographic matrices instead of one to the observation of the anomalies having the hyperthermic expression, allows for increased precision of the imaging of these anomalies through the prism of their thermal characteristics, because each sensor works only in the range of 1.5° C.

Existing solutions, including the reported inventions (Polish patent application No. P.381431 of the present applicant), insufficiently or incorrectly have solved the problem of correct identification of the nature of thermal anomalies, or by assuming too little gradient of the temperature difference (about 0.4° C.) for recognition of the observed anomalies for forecasting, inconsistently with clinical data, or through the adoption of a faulty temperature point of 36.6° C. for the division of thermographic scale for imaging of epidermic changes of hypo- and hyperthermic expression, which is in contradiction with the results of empirical measurements in vivo, or generally by not considering the possibility of the preliminary differentiation of that type of thermal anomalies during the test (no clear division of the hypo- and hyperthermic scale), which causes further objective difficulties in the interpretation of thermographic image from the medical point of view and can make the correct determination of the diagnosis impossible for the sake of the absence of correctly identified thermal range, within which thermographic tumour markers should be sought.

In the above-mentioned Polish patent application No. P.381431 an unsuccessful attempt to eliminate accidental separation of the operating ranges of the temperature was taken, in which the individual double sets of thermographic detectors had to work. However, the assumption in that invention that the separation point of thermographic scale for the detection and identification of pathological changes of the hypo- and hyperthermic nature on the surface of the tested breast, is the point of the physiological temperature of the human body set at 36.6° C. was incorrect, since in almost all empirical studies involving contact thermomastography or thermography by using infrared cameras, has not been established experimentally that the relatively constant physiological temperature of 36.6° C. measured under the patient's arm or even intraglandular, was transposed to equally stable and constant physiological temperature recorded on the surface of the examined breast. What's more, in the current research work on the breast thermography, including also the latest studies dedicated to the modelling of thermodynamic aspects of the breast cancer (see L. Jiang at al., Dynamic Characterization for Tumour and Deformation-Induced Thermal Contrasts on Breast Surface: A Simulation Study, Biomedical Applications in Molecular, Structural, and Functional Imaging, 2009), none functional dependence that allows to construct the appropriate functional dependence describing the transformation of the point intraglandular physiological temperature into the corresponding temperature of the breast surface in the conditions of the relative euthermy with the environment, causing no thermoregulatory response was identified. In particular, none of the scientific work stated that the physiological temperature of 36.6° C. measured under the patient's arm translates to the same standard temperature as measured on the breast surface. This means that indicated in this application and described by the present inventors the point of physiological separation of the detected thermal anomalies on these of the expression of hypo- and hyperthermic nature, was purely theoretical in nature and did not allow for the practical use in the apparatus for thermomastography. In the case of the contact thermography the issue of a distinct separation of thermal anomalies is crucial, because in the course of this study it is not possible to selectively eliminate the specific temperature ranges as passive infrared liquid crystal displays work in the factory established strict range of the temperatures, and therefore, despite the very high-resolution of the study, difficulties may arise in the normal visual evaluation of the characteristics of the observed changes in the temperatures, which as a consequence on that basis makes impossible to uniquely identify if the pathology is called “hot”—associated with the neoplastic processes and the mastitis type processes, or “cold”—the processes associated with mild, degenerative processes, especially when one set of displays is used for imaging of the temperature anomalies only in the double wide spectrum below or above 36.6° C.

Another known solution disclosed in U.S. patent application No. U.S. 20100312136A1, Ch. Cozzi in 2010, is a system comprising an apparatus and procedure solution, including the method of carrying out a medical diagnosis by means of thermography using the camera working at infrared.

The contact thermographic diagnosis based on the passive liquid crystal infrared displays and the electronic contact sensors for measuring the temperature, is fundamentally different from the remote thermography using the cameras working at infrared, primarily in terms of the physical effects used for obtaining the image of an isotherm distribution on the surface of the examined breast, as the first method is based on the transfer of the heat by conduction to the detector, while the second one is based on the emission. It is therefore not possible to directly compare these two methods, not only because the infrared mapping images generated by the apparatus according to the invention are formed at the passive display and are analogue, and the same testing and reading of the results takes place in real time, while a device according to the U.S. patent application No. U.S. 20100312136A1, Ch. Cozzi, generates the digital images, which are just produced with a small time shift by the computer as a result of the numerical analysis of the data obtained from the optoelectronic transducer receiving an infrared radiation, and the basis for assessing thermogram obtained in this manner is a quantitative analysis. Moreover, a recording of infrared mapping images from the apparatus according to the present invention is carried out by means of a separate, removable recording unit comprising a digital camera with a CCD transducer working in the visible light range of the electromagnetic wavelength range of about 380 to 700 nm, while the operating band of an infrared camera is in the range of so called near infrared from 700 to 1000 nm and it should be noted that an increase of the sensitivity does not apply for the classical cameras, such as those used in the present invention here described, because they have an infrared filter arranged in front of CCD transducer, which almost completely absorbs the radiation in the near-infrared band. Thus, the infrared mapping image coming from the camera of the present invention is the image generated as a result of the projection of a light that is selectively reflected in a thermotropic mesophase contained in the same display in the range of the visible spectrum, whereas the image obtained from the infrared camera is a picture completely reconstructed in a digital manner by a computer, as it is derived from the registration of an electromagnetic radiation invisible to the human eye. These two fundamental differences make both that the same basic mechanism of detection of the surface temperature of the breast for both inventions is different, and further the method of the formation of the infrared mapping image carrying the substantial medical information for the researcher is different, and as a consequence the technique of fixing the two images is different—in the first case there is a digitalisation of the analogue image, and in the other from the very beginning there is a reconstructed digital image.

Additionally, the U.S. patent application 20100312136A1 claims an algorithm comprising the procedure for making the thermographic digital images in two series and their comparison, in addition to the registration device through the attachable module, optional a network. Moreover, in said application the method of the identification of the locoregional hypo- or hyperthermia recorded on the surface of the examined breast, representing the essence of the intraglandular pathology is not clarified, in other words, Ch. Cozzi, neither in the description of his invention nor in the claims does indicate any specific point or the range of the temperatures that separate the organ normothermy from the thermal anomalies.

The novelty introduced by the present invention is an establishment of a completely different scope of the analysis of the thermal anomalies recordable by the method of the contact thermography on the surface of the examined breast, which is crucial for the medical meaning of such examination, because its purpose is not the multi-point temperature measurement itself, but to separate the anomalous areas with significant distinction of at least 0.5° C. in order to eliminate the random artefact thermal variations unrelated to the intraorganic hypo- or hyperthermia. Therefore, a new three-interval thermographic range related to the subject of the present invention, comprising in the range from 31.8° C. to 34.8° C., is further separated out into three sub-ranges: the first from 31.8° C. to 32.8° C. allowing to reveal the temperature anomalies on the surface of the tested breast showing the hypothermic expression, the second from 32.8° C. to 33.8° C. allowing to reveal the temperature anomalies on the surface of the examined breast showing the hyperthermic expression having a lower average temperature, and the third from 33.8° C. to 34.8° C. allowing to reveal the temperature anomalies of the surface of the examined breast showing the hyperthermic expression of a higher average temperature, constitutes the diagnostic value of the entire device and is the result of discoveries made by the present inventors by empirical studies in vivo with the patients.

Therefore, the inventors introduce in the present invention specifically defined reference temperature ranges within which the anomalies showing the hypothermic expression and the anomalies showing the hyperthermic expression are separately revealed. Otherwise, Also, these two inventions solve the problem of the calibration of the temperature detectors in completely different way, because in case of the Ch. Cozzi invention it is calibrated before each measurement, and in the case of the present invention, the calibration is done only once, at the stage of production of the liquid crystal infrared mapping displays, through the selection of the appropriate compositions of the mixtures of the chemical compounds selected from the group of chiral and non-chiral nematics to ensure a constant preset thermochromic response.

Another key difference between the present invention and the application cited above by Ch. Cozzi, is a method of reading the results of the thermomastographic examination, where the U.S. patent application No. 20100312136A1 employs the algorithm to automatically perform qualitative and quantitative analysis, and then automatically generate the scoring database separately based on the qualitative and quantitative analysis. The present invention has completely different assumption that thermograms rating is based on the binary criterion—positive/negative result including a decision support system with an artificial intelligence, which compares not individual series of the infrared mapping images obtained in the same patient, but the results of the study with other reference images of thermal anomalies collected in the database, resulting in a completely different analytical algorithm.

Thus, the object of the present invention is to provide a device for imaging, recording, and saving the thermographic image that allows correct identification of the nature of the thermal anomalies on the surface of the examined breast.

A further object of the invention is to provide a system of infrared mapping liquid crystal matrices suitable for use with the above-mentioned device to the detection of the pathological changes of hypo- and hyperthermic expression.

Another object of the invention is to provide a method of diagnosing breast pathology using the above equipment together with the infrared mapping liquid crystal matrices.

Thus, the present invention refers to the device for imaging, recording, and saving the thermographic image of the breast, which includes the image recorder and the infrared mapping liquid crystal matrix operated at the temperature range of approximately 1° in the range from 31.8° C. to 34.8° C., wherein the recorder preferably includes a housing, a light source, a camera and an optoelectronic transducer, an analog-to-digital converter, a power supply and a storage medium.

Preferably, the device according to the present invention operates on a three-interval thermographic scale comprising a detection range of a surface temperature of the breast from 31.8° C. to 34.8° C., and divided into three sub-ranges:
a first sub-range from 31.8° C. to 32.8° C. with the thermo-optical separation of 0.5° C., for the detection of the anomalies of the hypothermic expression,
a second sub-range from 32.8° C. to 33.8° C. with the thermo-optical separation of 0.5° C., for the detection of the anomalies of the hyperthermic expression of a lower temperature,
a third sub-range from 33.8° C. to 34.8° C. with the thermo-optical separation of 0.5° C., for the detection of the anomalies of the hyperthermic expression of a higher temperature.

Preferably, the infrared mapping liquid crystal matrix has a hand grip and an infrared mapping display, wherein the hand grip is made of plastic, in particular polypropylene.

Preferably, the infrared mapping matrix liquid crystal is round and has a diameter of at least 140 mm.

Preferably, the infrared mapping matrix liquid crystal operates in the temperature range from 31.8° C. to 32.8° C. with the thermo-optical separation of 0.5° C.

Preferably, the infrared mapping matrix liquid crystal operates in the temperature range from 32.8° C. to 33.8° C. with the thermo-optical separation of 0.5° C.

Preferably, the infrared mapping matrix liquid crystal operates in the temperature range from 33.8° C. to 34.8° C. with the thermo-optical separation of 0.5° C.

Preferably, the light source is at least one LED diode, especially one that emits a white light.

Preferably, the system of the camera with an optoelectronic transducer constitutes a digital camera with a CCD matrix of ⅓″ type with a sensitivity of at least 0.5 lux, a resolution of at least 540 lines/inch, with lens having a maintenance of a brightness at F 1.2 level.

Preferably, the power source is a DC power system having a voltage from 1.5 V to 9 V.

Preferably, the storage medium is selected from the media, such as a hard drive, an optical drive such as CD, CD-R, CD-RW, DVD-type optical disc, Blu-Ray, HD-DVD, a memory card or USB flash drive, and the most preferably the storage medium is a memory card a flash eprom type with a capacity of at least 4 GB.

Preferably, the device according to the present invention further includes an element capable of transferring of the data through a wired or a wireless transmission.

Preferably, the element that makes the data transfer possible is the wireless transmitter using radio wave frequency of 2.5 GHz, working in a Bluetooth standard.

The present invention also provides the system of three infrared mapping liquid crystal matrices comprising
a first liquid crystal matrix operating in the temperature range from 31.8° C. to 32.8° C. with the thermo-optical separation of 0.5° C. for the detection of the anomalies of the hypothermic expression;
a second liquid crystal matrix operating in the temperature range from 32.8° C. to 33.8° C. with the thermo-optical separation of 0.5° C. for the detection of the anomalies of the hyperthermic expression;
a third liquid crystal matrix (of a confirmatory type) operating in the temperature range from 33.8° C. to 34.8° C. with the thermo-optical separation of 0.5° C. for the detection of the anomalies even warmer showing the hypothermic expression.

The present invention relates to the use of said system of the three infrared mapping liquid crystal matrices for the detection of thermal anomalies on the surface of the breast,
the first matrix for the detection of the anomalies of the hypothermic expression, preferably related to the intraorganic pathologies of a benign characteristics,
the second matrix for the detection of the anomalies of the hyperthermic expression, preferably pathologies associated with intraorganic pathologies of a hyperplastic characteristics, and
the third confirmatory matrix for the detection of the anomalies of hyperthermic expression, preferably intraorganic pathologies associated with the hyperplastic characteristics.

The present invention also provides the method of diagnosing of the thermal anomalies of the breast surface using said device for imaging, recording, and saving the thermographic image of the breast, characterised in that it comprises the sequence of steps of:
applying the infrared mapping liquid crystal matrix combined in a detachable manner with the recorder, with the infrared mapping display to the examined breast;
turning on the light source;
recording the colour image of the isotherms depicted on the infrared mapping matrix using the camera equipped with the optoelectronic transducer and the analog-to-digital converter for a period of up to 20 seconds;
recording the obtained digital video signal on the storage medium;
transferring the recorded digital video signal to a computer or a mobile device equipped with a database of thermographic images of the breast and an artificial intelligence system or having established connection with such database and the artificial intelligence system via an intranet or the Internet;
using the artificial intelligence system to carry out the analysis of the obtained infrared mapping images using the database of the thermographic images of the breast;
as a result of the analysis, obtaining a guidance as to the most likely outcome of the thermographic examination in a binary system (positive/negative).

Preferably, in the method according to the present invention, said sequence of steps is performed three times, by successively using infrared mapping liquid crystal matrices being comprised in the above mentioned system of the three infrared mapping liquid crystal matrices.

Preferably, in the method according to the present invention, the reading of the thermographic examination results is carried out in the binary system—positive/negative based on the presence or absence in the infrared mapping image of a hypothermia marker visible as a delimited area of a different colour to the dominant colour of the thermal background visible at the working area of the first passive contact infrared mapping liquid crystal display.

Preferably, in the method according the present invention for detection there is used the thermographic three-interval scale comprising the range of the surface temperatures of the breast from 31.8° C. to 34.8° C., and separated into three sub-ranges:
the first sub-range from 31.8° C. to 32.8° C. with the thermo-optical separation of 0.5° C., for the detection of the anomalies of the hypothermic expression,
the second sub-range from 32.8° C. to 33.8° C. with the thermo-optical separation of 0.5° C., for the detection of the anomalies of the hyperthermic expression at the lower temperature,
the third sub-range from 33.8° C. to 34.8° C. with the thermo-optical separation of 0.5° C., for the detection of the anomalies of the hyperthermic expression of the higher temperature.

The present invention, which is the subject of this application eliminates the shortcomings of the contact thermography of the breast previously used, due to the discovery and use of a new scale of the operating temperature from 31.8° C. to 34.8° C. intended to thermomastographic apparatus according to the present invention, which scale is also separated out to the three sub-ranges: the first from 31.8° C. to 32.8° C. with the thermo-optical separation of 0.5° C., that allows to reveal the temperature anomalies on the surface of the tested breast showing the hypothermic expression, the second from 32.8 to 33.8° C. with the thermo-optical separation of 0.5° C. that allows to reveal the temperature anomalies on the surface of the examined breast having the hyperthermic expression with the lower average temperature, and the third from 33.8° C. to 34.8° C. with the thermo-optical separation of 0.5° C. that allows to reveal the temperature anomalies on the surface of the examined breast having the hyperthermic expression with the higher average temperature.

In experimental studies on the breast thermography, the range of the temperatures recorded on the surface of the examined breast rarely was below 31° C. and above 35° C. (see J F Head, Determination of mean temperatures of normal breast and breast whole quadrants by infrared imaging and image analysis, IEEE, 2001), and therefore the above-mentioned Polish patent application No. P.381431 did not properly consider the experimentally established upper limit of the hypothermia and lower limit of the hyperthermia of the organs disclosed in the breast thermography as a point of division was there determined theoretically at 36.6° C. The consequence of such determination of the reference point of division of the thermographic scale in the above mentioned invention is that none of its two measurement sub-ranges, respectively, for the analysis of the anomalies having the hypothermic expression at the temperatures below 36.6° C., nor the anomalies having the hyperthermic expression at the temperatures above 36.6° C., do not enter in the appropriate detection ranges of the present invention, because they end for the interval for imaging of the hypothermic changes at 32.8° C., and for the range for imaging of the hyperthermic changes said sub-ranges start above 32.8° C. and end at 34.8° C., and are separated from the boundary value of 36.6° C. of as much as 1.8° C.

Thresholds of the temperature ranges determined empirically for the device according to the present invention, are not only a basis for the creation of a new thermographic scale designed exclusively for specialised use in the thermomastographic examination, which is important and critical breakthrough in terms of innovation, because none of the filed inventions so far relating to the devices for contact thermomastography, did not allow to carry out real-time classification of the thermal anomalies visualised in terms of their thermodynamic and pathophysiological characteristics of both, while allowing an initial diagnosis based on the presence of the markers of the hypo- or hyperthermia on the surface of the examined breast, which are predictors of the presence of a specific type of intraglandular pathology. The present invention therefore includes in particular the new thermographic scale, which, in this or a similar temperature range and in a specific, defined and three-interval functional division, has not been disclosed in any other published patent application in the field of thermomastography.

The device according to the present invention does not require the examination of both breasts simultaneously.

The solutions disclosed in the Polish patent application No. P.381431 and in the present description and claims are different in the key aspects, the first invention refers to the dual-band operating temperature scale separated around the point 36.6° C., and the other to the three- interval scale separated around points 32.8° C. and 33.8° C. Also in the technical context, the first invention consists of a double detection system comprising two matrices enabling the parallel differential diagnosis of the both breasts, and the set consists of two kits of differently calibrated matrices, while the apparatus according to the present invention consists of the system of the three passive infrared mapping liquid crystal displays placed in the separate hand grips, applied sequentially, first to one, then the other breast.

In the present invention the use of the thermo-optical separation of 0.5° C. is consistent with the data obtained from empirical observations [Zhao et al. 2008] and according to the scientific literature is sufficient to consider such temperature difference as significant from the point of view of the diagnostic and prognostic value, because the changes suspected of neoplastic transformation are characterised by the average thermal differentiation of 0.7° C. in ratio to the surrounding healthy tissue.

It was surprisingly found that the use of a triple set of the infrared mapping matrices, with a total thermal detection range covers an area of the hypo- and hyperthermic anomalies present on the surface of the examined breast, while maintaining the possibility of a separate observation (separation), allows for clear differentiation and thus, to a certain interpretation of the examination from the medical point of view.

The basis of the invention is a new division of the thermographic scale on the three complementary ranges, obtained from carried out empirical measurements. The application of this division makes possible to uniquely identify the thermographic markers of the benign and malignant processes. The observation of the thermographic markers of the benign process takes place on a single infrared mapping matrix, and for the observation of the markers of the malignant processes, the present invention provides two separate matrices, of which the last (third) acts as a confirmatory matrix. The disclosure on the third confirmatory matrix the thermal marker is equal to the confirmation of the presence of the outbreak of the hyperthermia, which is associated with the neoplastic processes. The above-mentioned Zhao at al. [2008] pointed out the statistical association of the thermal differentiation of 0.7° C. order and more, with the presence of the malignant tumour.

For the device according to the present invention there are programmed during the production of the thermographic matrices, three complementary operating temperature ranges, tailored to the experimentally determined thresholds in relation to the standard surface temperature of the breast of 32.8° C. The application of these thresholds allows for the filtration of the thermal background of the examined breast and eliminates the artefact readings, by cutting off, in the case of the first matrix, the thermal background in the lower range of the thermographic scale at 32.8° C., which allows for the visualisation of only the changes with the temperature of at least 0.5° C., compared to the standard surface temperature of the breast (characterised by the benign pathologies) and respectively for the second matrix, the cutting off the thermal background in the upper range of the thermographic scale at 32.8° C., which allows for the observation of the anomalies at the temperature higher by at least 0.5° C. in relation to the surface temperature of the normal breast (that is significantly warmer—hyperthermic, being characteristic of the malignant abnormalities), and for the third matrix, cutting off the thermal background was at even higher temperatures, that is above 33.8° C., which allows to reveal the anomalies that are significantly warmer of at least 1° C., relative to the standard breast surface temperature of 32.8° C. (obviously related to the thermal characteristics of the tumour with malignant course).

The triple system of the thermographic matrices is in the device of the present invention an unique application of the concept of thermal hardware filtration, not for the purpose of implementing of the technical purposes (to ensure appropriate scope for the thermal detection study of the breast in women), but only for a specific medical purpose, understood as a solution to the initial differentiation by type of the intraorganic pathology through its association with the thermal characteristics, forming specified thermographic marker.

Without determining whether the observed anomaly has the of hypo- or hyperthermic type of expression, no one can talk about the presence of the thermographic markers as the markers are attributed to the thermodynamic model of tumour growth and the formation of cysts.

The binary criterion for the evaluation of the examination result by the contact thermography filtered by using the device according to the present invention, it is possible to use, because each of three passive infrared mapping liquid crystal displays included in the diagnostic kit, works in a strictly defined and programmed at the manufacturing stage temperature range, correlated with the new thermographic scale, so that in given temperature range there is a search of specific types of mammary pathology. This means that the individual passive infrared mapping liquid crystal displays create a set of functional tests aimed at the detection of functional thermodynamic markers specific only for a group of breast pathology and giving a fixed expression of heat, which makes that on the display designed for the imaging of the hyperthermic expression, researcher will not see the signs corresponding to the benign pathologies associated with the degenerative processes, for example fluid cysts, and vice versa. The binary criterion boils simply to identification of the presence of the optically delimited anomaly, determined in relation to dominant thermal background expressed on the display by the predominant colour of the thermo-optical response of the thermotropic mesophase, because each primary colour visible on the display in the RGB system (red-green-blue) corresponds to the difference in temperature of 0.5° C. order. In order to support this binomial assessment it is also possible to specify the quantitative scope, based on relational, fixing the percentage of the active surface of the infrared mapping imaging display occupied by the given, dominant colour corresponding to the specific temperature of the thermal background of the examined organ in relation to the visualised, demarcated colour anomaly, which should include a minimum surface. It should be noted that the quantitative determinants are not the only criterion of the evaluation and qualification of the contact thermogram as abnormal or normal, because the test is being able to detect even the smallest expressions of the anomalous temperatures on the surface of the tested breast, but can give additional information about the scale of severity of the pathological process. It was experimentally established that the mere presence of the thermal marker identified on any passive infrared mapping liquid crystal display according to the present invention can classify the whole test by contact thermography as giving positive result, indicating the thermodynamic characteristics of the observed anomaly, hence the quantitative parameters is associated with the marker is not the subject of the claims in the present patent application.

Each of the thermographic matrices in order to act as a fully functional thermographic detector requires being mounted in the hand grip made of a flexible unbreakable plastic, preferably polypropylene.

Each of the matrices is preferably round and has a diameter of at least 140 mm.

In addition, the device according to the present invention is equipped with the optoelectronic recorder of the colour images of the isothermal distribution on the surface of the examined breast being revealed on the passive infrared mapping matrices, consisting of the digital camera system, preferably with the CCD (Charge Coupled Device) matrix, allowing the recording, and then the reading of the electric signal that is proportional to the amount of light falling on it and the electronic system enabling the analog-to-digital conversion of the image and then encoding the video signal, for example, according to the standard ISO/IEC 14496-10:2004 (H.264/AVC), the storage medium, such as flash memory card, allowing for the digital recording of the entire test sequence and preferably a wireless transmitter that contains the stored video image, working, for example, in the standard according to the specification IEEE 802.15.1 that uses radio waves in the 2.4 GHz ISM band (Bluetooth). The recorder dramatically expands the diagnostic potentials of the tester by providing the ability to send the full sequence of the thermographic examination as a digital file to the computer and further by means of the Internet using the analytic centre in order to carry out its thorough analysis with the use of the algorithm based on the artificial intelligence system comparing the normal and the pathological patterns of the thermograms with those actually obtained during a particular test. The support of the thermograms analysis by the artificial intelligence system using different algorithms to compare different images increases the accuracy of the examination, facilitating the identification of the important from a medical point of view the thermal anomalies, which has a significant impact on the confidence of the diagnosis.

The recorder of the present invention is an additional, complete and removable component with its own power supply, which is secured by a system of bolts into the plastic hand grip of the infrared mapping matrix.

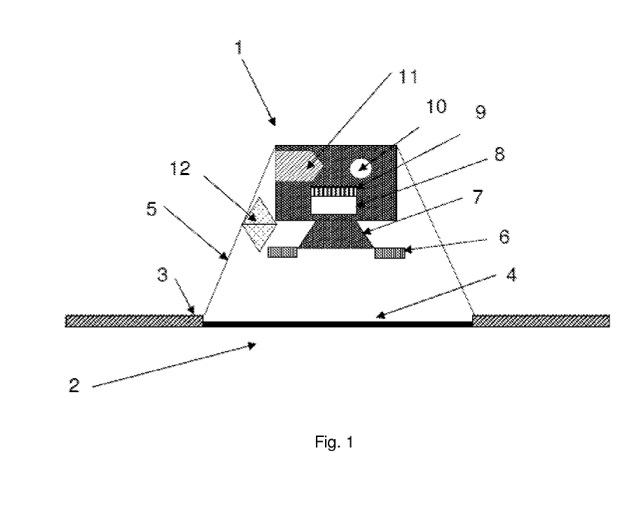

The embodiment of the device according to the present invention in shown in the drawings, in which FIG. 1 illustrates schematically the construction of the recorder in combination with the infrared mapping liquid crystal matrix display.

In the FIG. 1 the recorder having the casing 5 is composed of five basic elements, such as:
the digital micro camera 7 with the optoelectronic transducer 8, which is a part of the CCD matrix having a minimum size of ⅓″, with the sensitivity of at least 0.5 lux, the resolution of at least 540 lines/inch, and with lens having a maintenance of a brightness at F 1.2 level;
the storage medium 11, such as the memory card the flash eprom type with the capacity of at least 4 GB;
the light source 6, which is a distributed lighting system with LED diodes emitting the white light, arranged around the lens of the camera 7 directed on the infrared mapping display 4 of the liquid crystal matrix 2;
the own DC power supply system 10, in this case 9V, and
the component for the data transmission 12, here the wireless transmitter that uses radio waves with a frequency of 2.4 GHz, working in the Bluetooth standard.

The recorder 1 according to the present invention is attached to the hand grip 3 of the infrared mapping liquid crystal matrix 2.

The purpose of the recorder is to carry out the real-time video recording of the full sequence of the thermographic examination of the breast, which takes about 15 seconds from the time of the correct application of the detector to the examined breast. The holding of the detector in one place of the examined breast that lasts longer than 20 seconds, may lead to the equalisation of the infrared mapping matrix 2 temperatures and the examined breast area, leading as a result to the loss or distortion of the thermographic image.

The recorder 1 according to the present invention is activated manually by the person performing the test, and automatically turns off after 15 seconds, which is indicated by turning on the red LED diode on the body 5 and a single beep. The method of diagnosing the breast pathology using this device includes the steps, wherein the infrared mapping liquid crystal matrix display combined in a detachable manner with the recorder 1 is applied with the infrared mapping display 4 to the examined breast, then the light source 6 is automatically turned on and for a period of up to 20 seconds the colour image of the isotherms depicted on the infrared mapping matrix is recorded using the digital micro camera 7 with the CCD matrix 8 and the analog-to-digital converter 9. The resulting digital video signal is then recorded in the flash memory card 11, and said sequence of the image recording and saving is carried out for three times, using sequentially the three infrared mapping liquid crystal matrices 2 operating in the three examined temperature ranges. After the examination is completed, the data stored in the flash memory card 11 are transmitted by pressing a single button by the Bluetooth transmitter 12 to the computer equipped with the Bluetooth receiver, having a connection with the database of the thermographic images of the breast and the artificial intelligence system via the Internet, to analyse the obtained infrared mapping images obtained using the database of the thermographic images of the breast. As a result of the analysis the guidance as to the most likely outcome of the thermographic examination in the binary system (positive/negative) is obtained.

The role of the passive infrared mapping detector in the infrared mapping liquid crystal matrix 2 is performed by the set of the three heat-sensitive matrices 2 containing thermotropic liquid crystals from the group of chiral nematics.

Each of the matrices 2 is cut in a circular shape with a diameter of 140 mm and is mounted in the plastic hand grip 3.

To produce the thermotropic mesophase being a key heat-sensitive element—the infrared mapping display 4 of each of the three thermographic matrices 2, a mixture of liquid crystal compounds according to the invention, which is the subject of the Polish patent application No. P.390320 was used, having the following compositions.

The mixture of the first matrix (composition by weight):

| | |
|---|---|
| cholesteryl pelargonate | 48.61% |
| cholesteryl oleyl carbonate | 50.39% |
| cholesteryl propionate | 0.28% |
| cholesteryl chloride | 0.20% |
| 4,4′-dipentylazoxybenzene | 0.52%. |

For this mixture the mesophase is thermo optically responsive in the temperature range from 31.8° C. to 32.8° C., wherein at the temperature of 31.8° C. a reflected in the mesophase light of red colour (of wavelength 720 nm) appears and this colour is maintained through 0.5° C., then at the temperature of 32.3° C. a reflected in the mesophase light of green colour (of wavelength 545 nm) appears and this colour is maintained through 0.5° C., and at the temperature of 32.8° C. a reflected in the mesophase light of blue colour (of wavelength 410 nm) appears and this colour is maintained through 0.5° C.

The mixture of the second matrix (composition by weight):

| | |
|---|---|
| cholesteryl pelargonate | 50.28% |
| cholesteryl oleyl carbonate | 48.72% |
| cholesteryl propionate | 0.24% |
| cholesteryl chloride | 0.18% |
| 4,4′-dipentylazoxybenzene | 0.58% |

For this mixture the mesophase is thermo optically responsive in the temperature range from 32.8° C. to 33.8° C., wherein at the temperature of 32.8° C. a reflected in the mesophase light of red colour (of wavelength 720 nm) appears and this colour is maintained through 0.5° C., then at the temperature of 33.3° C. a reflected in the mesophase light of green colour (of wavelength 545 nm) appears and this colour is maintained through 0.5° C., and at the temperature of 33.8° C. a reflected in the mesophase light of blue colour (of wavelength 410 nm) appears and this colour is maintained through 0.5° C.

The mixture for the third matrix (composition by weight):

| | |
|---|---|
| cholesteryl pelargonate | 52.53% |
| cholesteryl oleyl carbonate | 46.47% |
| cholesteryl propionate | 0.18% |
| cholesteryl chloride | 0.16% |
| 4,4′-dipentylazoxybenzene | 0.66% |

For this mixture the mesophase is thermo optically responsive in the temperature range from 33.8° C. to 34.8° C., and at the temperature of 33.8° C. a reflected in the mesophase light of red colour (of wavelength 720 nm) appears and this colour is maintained through 0.5° C., then at the temperature of 34.3° C. a reflected in the mesophase light of green colour (of wavelength 545 nm) appears and this colour is maintained through 0.5° C., and at the temperature of 34.8° C. a reflected in the mesophase light of blue colour (of wavelength 410 nm) appears and this colour is maintained through 0.5° C. Then, each of the hand grips 3 containing the infrared mapping matrix 2 is connected in the detachable manner with the real-time optoelectronic recorder of the video image, allowing for the digital recording of the sequence of the thermographic examination of the breast and its transmission by the wireless Bluetooth module to the external device—the computer or other mobile device.

1-21. (canceled)

22. A device for imaging, recording, and saving a thermographic image of a breast, comprising:
an image recorder and an infrared mapping liquid crystal matrix working in a temperature range of approximately 1° in a range from 31.8° C. to 34.8° C., wherein the recorder includes a housing, a light source, a camera with an optoelectronic transducer, an analog-digital converter, a power source and a storage medium.

23. The device according to claim 22, wherein operation occurs in a three-interval thermographic scale comprising the range of the surface temperature detection of the breast from 31.8° C. to 34.8° C., wherein the range is divided:
a first sub-range from 31.8° C. to. 32.8° C. with a thermo-optical separation of 0.5° C., for detection of anomalies of a hypothermic expression;
a second sub-range from 32.8° C. to 33.8° C. with a thermo-optical separation of 0.5° C., for detection of anomalies of a hyperthermic expression at a lower temperature, and
a third sub-range from 33.8° C. to 34.8° C. with a thermo-optical separation of 0.5° C., for detection of anomalies of the hyperthermic expression of a higher temperature.

24. The device according to claim 22, wherein the infrared mapping liquid crystal matrix has a hand grip and an infrared mapping display, wherein the hand grip is made of plastic.

25. The device according to claim 22, wherein the infrared mapping liquid crystal matrix is circular and has a diameter of at least 140 mm.

26. The device according to any one of claim 22 wherein the infrared mapping liquid crystal matrix operates in a temperature range from 31.8° C. to 32.8° C. with a thermo-optical separation of 0.5° C.

27. The device according to claim 22, wherein the infrared mapping liquid crystal matrix operates in a temperature range from 32.8° C. to 33.8° C. with a thermo-optical separation of 0.5° C.

28. The device according to claim 22, wherein the infrared mapping liquid crystal matrix operates in a temperature range from 33.8° C. to 34.8° C. with a thermo-optical separation of 0.5° C.

29. The device according to claim 22, wherein the light source is at least one LED diode.

30. The device according to claim 29, wherein the LED diode emits a white light.

31. The device according to claim 22, wherein the camera system with the optoelectronic transducer is a digital camera with a CCD sensor of ⅓″ type with a sensitivity of at least 0.5 lux, and a resolution of at least 540 lines I inch, with lens having a maintenance of a brightness at F 1.2 level.

32. The device according to claim 22, wherein said power source is a DC power supply system at a voltage of between 1.5 V to 9 V.

33. The device according to claim 22, wherein the storage medium is one of a hard drive, optical drive, Blu-Ray, HD-DVD, a memory card, or memory USB type.

34. The device according to claim 22, wherein the storage medium is a memory card of a flash eprom type having minimum capacity of 4 GB.

35. The device according to claim 22, further comprising a data transmission device for transmitting data through a wired or wireless transmission.

36. The device according to claim 35, wherein the data transmission device is a wireless transmitter that uses radio waves of frequency 2.5 GHz, working in the Bluetooth standard.

37. A system of three infrared mapping liquid crystal matrices, comprising:
a first liquid crystal matrix operating in a temperature range from 31.8° C. to 32.8° C. with the thermo-optical separation of 0.5° C. that detects anomalies of hypothermic expression;
a second liquid crystal matrix operating in a temperature range from 32.8° C. to 33.8° C. with a thermo-optical separation of 0.5° C. that detect anomalies of hyperthermic expression;
a third liquid crystal matrix operating in a temperature range from 33.8° C. to 34.8° C. with a thermo-optical separation of 0.5° C. that detects even warmer anomalies showing hypothermic expression.

38. The system of three infrared mapping liquid crystal matrices of claim 37, wherein the liquid crystal matrices detect thermal anomalies on a surface of a breast, and wherein
the first matrix detects anomalies of a hypothermic expression related to the intraorganic pathologies of a benign characteristics,
the second matrix detects anomalies of a hyperthermic expression, associated with intraorganic pathologies of a hyperplastic characteristic, and
the third confirmatory matrix detects anomalies of hyperthermic expression, associated with a proliferative nature.

39. A method of diagnosing of the thermal anomalies of the breast surface using the device for imaging, recording and saving thermographic image of the breast according to the claim 22, comprising a sequence of steps of:
applying the infrared mapping liquid crystal matrix combined in a detachable manner with the recorder, with the infrared mapping display to the examined breast;
turning on the light source;
recording the colour image of the isotherms depicted on the infrared mapping matrix using the camera equipped with the optoelectronic transducer and the analog-to-digital converter for a period of up to 20 seconds;
recording the obtained digital video signal on the storage medium;
transferring the recorded digital video signal to a computer or a mobile device equipped with a database of thermographic images of the breast and an artificial intelligence system or having established connection with such database and the artificial intelligence system via an intranet or the internet;
using the artificial intelligence system to carry out the analysis of the obtained infrared mapping images using the database of the thermographic images of the breast;
obtaining a guidance as to the most likely outcome of the thermographic examination in a binary system as a result of the analysis.

40. The method according to claim 39, wherein said sequence of the recording and saving the image is performed three times, by successively using three infrared mapping liquid crystal matrices.

41. The method according to claim 39, wherein the reading of the thermographic examination result is carried out in the binary system based on the presence or absence in the infrared mapping image of a hypothermia marker visible as a delimited area of a different colour in relation to a dominant colour of the thermal background visible at the working area of the first passive contact infrared mapping liquid crystal display.

42. The method according to claim 39, wherein for detection there is used the thermographic three-interval scale comprising a range of surface temperatures of a breast from 31.8° C. to 34.8° C., and separated into three sub-ranges, wherein
a first sub-range from 31.8° C. to 32.8° C. with a thermo-optical separation of 0.5° C., for the detection of anomalies of a hypothermic expression,
a second sub-range from 32.8° C. to 33.8° C. with a thermo-optical separation of 0.5° C., for the detection of anomalies of a hyperthermic expression of a lower temperature,
a third sub-range from 33.8° C. to 34.8° C. with a thermo-optical separation of 0.5° C., for a detection of anomalies of a hyperthermic expression of a higher temperature.
